(19) United States
(12) Patent Application Publication
(10) Pub. No.: US 2026/0209156 A1
(43) Pub. Date: Jul. 23, 2026

(54) CONCENTRATION OF AQUEOUS ACETIC ACID BY CLATHRATE HYDRATE FORMATION

(71) Applicant: NOVA CHEMICALS (INTERNATIONAL) S.A., Fribourg (CH)

(72) Inventors: Bolaji OLAYIWOLA, Calgary (CA); Vasily SIMANZHENKOV, Calgary (CA); Shahin GOODARZNIA, Calgary (CA)

(21) Appl. No.: 19/139,045

(22) PCT Filed: Dec. 12, 2023
(86) PCT No.: PCT/IB2023/062562

§ 371 Date: Jun. 13, 2025

Related U.S. Application Data
(60) Provisional application No. 63/432,738, filed on Dec. 15, 2022.

(51) Int. Cl. C07C 51/43

(52) U.S. Cl. CPC C07C 51/43

(57) ABSTRACT

A method and a system for concentrating an acetic acid solution are provided An exemplary system includes a hydrate reactor to form solid hydrates in the acetic acid solution, a filtration system to remove the solid hydrates formed in the hydrate reactor, and a drying system to separate ethane from water.

### TECHNICAL FIELD

The present disclosure is directed to concentrating acetic acid from a waste stream by the formation of clathrate hydrates.

### BACKGROUND ART

Selective oxidation processes, such as oxidative dehydrogenation (ODH), are an alternative to steam cracking that are exothermic and produce little or no coke. In ODH, a lower alkane, such as ethane, is mixed with oxygen in the presence of a catalyst and optionally an inert diluent, such as carbon dioxide or nitrogen or steam, which may be performed at temperatures as low as 300° C., to produce the corresponding alkene. Various other oxidation products may be produced in this process, including carbon dioxide and acetic acid, among others. Acetic acid is a valuable organic compound. It is a precursor to produce different chemicals used in textiles manufacturing, ink formulations, polymer manufacturing, and the like. In many chemical processes, including ODH, any acetic acid generated is dilute and often requires dehydration to form a useful product. The method of concentrating aqueous acetic acid is generally liquid-liquid extraction process using MTBE, ethyl acetate, and the like. However, liquid-liquid extraction adds significant costs from the use of additional solvents and purification steps.

### SUMMARY OF INVENTION

An implementation described in examples herein provides a method for concentrating acetic acid. The method includes adding ethane to an acetic acid solution, controlling temperature and pressure to form a mixture of solid hydrates in the acetic acid solution, and separating the solid hydrates from the acetic acid solution to form a more concentrated acetic acid solution.

Another implementation described in examples herein provides a system for concentrating an acetic acid solution. The system includes a hydrate reactor to form solid hydrates in the acetic acid solution, a filtration system to remove the solid hydrates formed in the hydrate reactor, and a drying system to separate ethane from water.

### BRIEF DESCRIPTION OF THE DRAWINGS

FIG. 1 is a block diagram of an oxidative dehydrogenation (ODH) system for the production of ethylene from the selective oxidation of ethane, in the presence of water.

FIG. 2 is a schematic diagram of an ODH system that integrates a system for using hydrates to increase the concentration of an acetic acid stream.

FIG. 3 is a schematic diagram of a process for continuously forming hydrates to concentrate the acetic acid.

FIG. 4 is a schematic diagram of a process for the concentration of acetic acid by the continuous formation and removal of clathrate hydrates.

FIG. 5 is a process flow diagram of a method for concentrating acetic acid using the formation of clathrate hydrates.

FIG. 6 is a process flow diagram of a method for integrating the concentration of acetic acid using the formation of clathrate hydrates with an oxidative dehydrogenation process.

FIG. 7 is a simulation flowsheet for simulating the feed going into the clathrate hydrates unit using ASPEN Plus® V11.

FIG. 8 is a plot of the resulting P-T diagram for the incipient hydrate formation.

### DESCRIPTION OF EMBODIMENTS

A process for concentrating aqueous acetic acid is disclosed herein. Small molecules such as methane, ethane, carbon dioxide, hydrogen sulfide, oxygen, nitrogen, and hydrogen, among others, form clathrate hydrate with water at suitable temperature and pressure. Generally, during the formation of the solid hydrates, larger molecules, such as the acetic acid, are excluded from the matrix. As the solid hydrates are filtered out, the concentration of the acetic acid is increased.

As described herein, the process can be integrated with an oxidative dehydrogenation system. For example, the aqueous acetic acid condensed in a stripper, or quench tower, downstream of the ODH reactor is mixed with the ethane from bottom of the C2-splitter. This mixture is sent to a hydrate formation reactor that is operated at lower temperatures and high pressure. The mixture is continuously agitated and cooled to the desired temperature to initiate hydrate formation. The more concentrated aqueous acetic acid solution is filtered to remove the solid hydrate. If the aqueous acetic acid from the quench tower has an acetic acid concentration of about 1-2 wt. %, it may useful to recycle the more concentrated acetic acid solution to the hydrate unit to continue to increase the concentration.

The use of clathrate hydrate formation for the concentration of acetic acid was demonstrated by simulation using ASPEN HYSYS. The results are discussed further with respect to the examples.

Other than in the operating examples or where otherwise indicated, all numbers or expressions referring to quantities of ingredients, reaction conditions, etc. used in the specification and claims are to be understood as modified in all instances by the term “about”. Accordingly, unless indicated to the contrary, the numerical parameters set forth in the following specification and attached claims are approximations that can vary depending upon the desired properties, which the present disclosure desires to obtain. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the disclosure are approximations, the numerical values set forth in the specific examples are reported as precisely as possible. Any numerical values, however, inherently contain certain errors necessarily resulting from the standard deviation found in their respective testing measurements.

In addition, it should be understood that any numerical range recited herein is intended to include all sub-ranges subsumed therein. For example, a range of “1 to 10” is intended to include all sub-ranges between and including the recited minimum value of 1 and the recited maximum value of 10; that is, having a minimum value equal to or greater than 1 and a maximum value of equal to or less than 10. Because the disclosed numerical ranges are continuous, they include every value between the minimum and maximum values. Unless expressly indicated otherwise, the various numerical ranges specified in this application are approximations.

As used herein, the term, “selective oxidation” or “SO” refers to an oxidation process that does not proceed to complete thermodynamic oxidation, for example, stopping at products more complex than carbon dioxide and water. As used herein, “oxidative dehydrogenation” or “ODH” is a subset of selective oxidation, and refers to processes that couple the endothermic dehydrogenation of an alkane with the strongly exothermic oxidation of hydrogen as is further described herein.

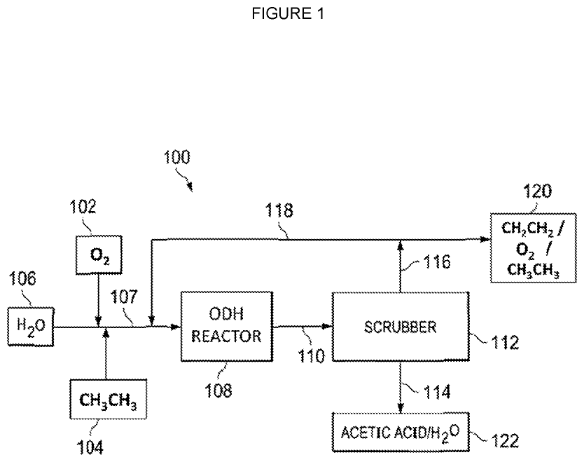

FIG. 1 is a block diagram of an oxidative dehydrogenation (ODH) system 100 for the production of ethylene from the selective oxidation of ethane, in the presence of oxygen. It can be understood that this provides one system for producing a dilute solution of acetic acid. In other examples, a dilute solution of acetic acid is provided as a direct product from the selective oxidation of methane in the presence of water.

In this example, the feedstocks include oxygen (O₂) 102, and ethane (C₂H₆) 104. In some examples, the ethane 104 may be replaced with other light hydrocarbons, such as methane, among others. In some embodiments, water 106 is added as an inert diluent, for example, to lower the flammability of an oxygen/hydrocarbon mixture. The feedstocks 102 and 104, and the water 106 are added to an ODH reactor 108 as a feed stream 107 for conversion of at least a portion of the feedstocks to ethylene and byproducts, such as acetic acid. The feedstocks may be added as individual feed streams, or in combined feed streams with each other and recycled feedstock streams. Further, other feedstocks, such as carbon dioxide, may be added to the system in addition to or in place of the feedstocks mentioned here.

The H₂O 106, when used as inert diluent, is generally added as steam to prevent thermal shock from damaging the catalyst bed. The amount of steam added to the ODH reactor 108 may be adjusted to control the selectivity for the production of ethylene versus acetic acid. Generally, higher amounts of water added increase the amount of acetic acid formed in the reaction.

The ethane 104 may be provided from another hydrocarbon process, such as separation in a gas plant. As described herein, the light hydrocarbon is not limited to ethane. In some examples, other light hydrocarbons, such as, propane, or others, may be used in a light hydrocarbon feed stream to the ODH reactor 108. For either the ethane or the carbon dioxide, a pipeline may be used to source a portion of the feedstock if insufficient amounts are being provided from a usual source.

Formation of acetic acid during the ODH process can occur by the reaction of carbon dioxide and ethane, as shown in equation 1.

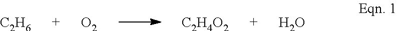

The reaction is facilitated by an ODH catalyst. In some examples, the ODH catalyst has the formula shown in equation 2.

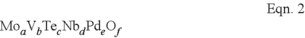

In this formula a, b, c, d, e, and fare relative atomic amounts of the elements Mo, V, Te, Nb, Pd, and O, respectively. When a is 1, b is between 0.01 and 1.0, c is between 0 and 1.0, d is between 0 and 1.0, e is between 0 and 0.10, and f is a number to at least satisfy the valence state of any metal elements present in the ODH catalyst. Another example of a catalyst that may be used in processes is shown in the formula of equation 3.

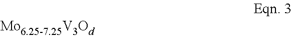

In equation 3, d is a number to at least satisfy the valence of the any present metal elements in the catalyst. In other examples, the ODH catalyst includes vanadium in addition to any other components, such as mixed metal oxides, that are present.

As used herein, the phrase “to at least satisfy the valence” indicates that additional oxygen may be present. In some embodiments, oxygen is absorbed into the catalyst as lattice oxygen. The lattice oxygen may participate in the catalytic reactions through the transfer of oxygen to the hydrocarbon substrate. This temporarily generates a vacancy, or defect, in the catalyst that is replenished by other oxygen atoms or molecules absorbed in the lattice or external to the catalyst. Accordingly, the catalyst can rely on the ability of the metal oxide to form phases that are not stoichiometric to promote reactions.

In some embodiments, oxygen may be present on the surface of the catalyst in the form of hydroxyl groups. In these embodiments, the amount of oxygen for at least a portion of the metals present is double the amount needed to satisfy the valences of the metals, as a hydrogen atom is also present.

Accordingly, there is generally more oxygen in the catalyst than is required to satisfy the valences of the metals. The measurement of the oxygen may be performed by any number of different techniques. The oxygen present beyond that needed to satisfy the valence states is a combination of lattice oxygen and hydroxyl groups, as well as any other oxygen containing groups, such as carbonate groups and the like.

In some examples, the catalyst may be supported on, or agglomerated with, a carrier. Some examples of carriers include acidic, basic, or neutral binder slurries of TiO₂, ZrO₂, Al₂O₃, AlO(OH) and mixtures thereof. Another useful carrier includes Nb₂O₅. The agglomerated catalyst may be extruded in a suitable shape, such as rings, spheres, saddles, cylinders, and the like of a size typically used in fixed bed reactors. When the catalyst is extruded, various extrusion aids known in the art can be used. In some cases, the resulting support may have a cumulative surface area, as measured by BET, of less than about 35 m²/g, less than about 20 m²/g, or less than about 3 m²/g, and a cumulative pore volume from about 0.05 to about 0.50 cm³/g.

The reactor effluent 110 is sent to a scrubber 112, for the removal of acetic acid, other oxygenates, and water. The scrubber 112 may include any number of configurations, such as a heat exchanger followed by a flash vessel or quench tower, which condenses liquids 114 formed in the ODH reactor 108 and separates them from gases 116. At least a portion of the gases 116 can be returned to the ODH reactor 108 to be combined with the feed stream 107. In some embodiments, increasing the fraction of the recycle feed 118 that is returned to the ODH reactor 108 may increase the total conversion of the O₂ 102 and C₂H₆ 104 to acetic acid, if desirable. In examples, the conversion rate of O₂ 102 and ethane 104 in a single pass through the ODH reactor 108 is about 15 wt. %, 10 wt. %, 5 wt. %, or lower, depending on the flow rate through the ODH reactor 108.

The gases separated from the liquids 114 include unreacted O₂ and ethane, which may be returned to the ODH reactor as a recycle feed 118 that is combined with one or more of the feedstocks 102 or 104. In some examples, the gases 116 are provided as a mixed product 120 stream to other processes, such as cryogenic separation to isolate excess ethane 104 for use in the acetic acid purification process. The liquids 114 include dilute acetic acid 122, which may be purified, as described herein, to increase the concentration of the acetic acid, for example, for sale or use. In some examples, at least a portion of the water removed during the purification of the acetic acid may be returned to the process feed, for example, being combined with the H₂O 106.

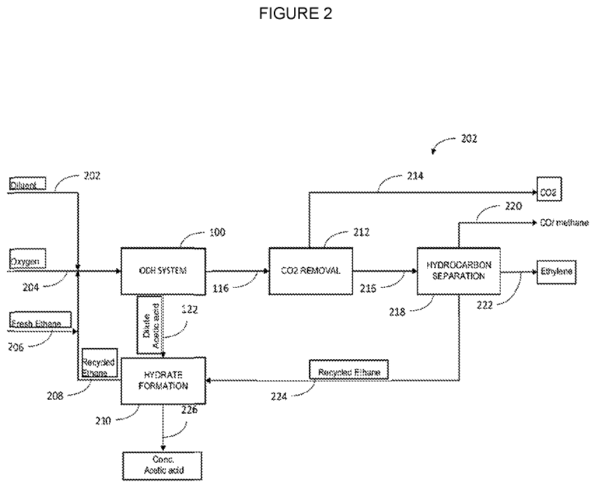

FIG. 2 is a schematic diagram of an ODH system 200 that integrates a system for using hydrates to increase the concentration of an acetic acid stream. Like numbered items are as described with respect to FIG. 1. In this example, the feedstocks to the oxidative dehydrogenation system 100 include a diluent 202, oxygen 204, fresh ethane 206, and recycle ethane 208. The diluent 202 is used to lower the flammability during the mixing of the feedstocks prior to the introduction to the ODH reactor 108 (FIG. 1), for example, in a separate mixing vessel in the ODH system 100. However, the diluent 202 may participate in the ODH reaction, for example, such as carbon dioxide. From the ODH system 100, the dilute acetic acid 122 is fed to a hydrate formation process 210. In various examples, the dilute acetic acid 122 has a concentration of about 1.5% acetic acid in water, about 3% acetic acid water, about 5% acetic acid in water, or greater. The concentration of the acetic acid depends on the operation of the ODH system 100. For example, and ODH system that is specifically tuned to the production of the ethylene 222 will produce less amounts of acetic acid.

The gases 116 are fed to a CO₂ removal process 212. In the CO₂ removal process 212, a CO₂ stream 214 is separated from the gases 116, for example, in an amine scrubber. Other processes may be used for the CO₂ removal process 212, such as a cryogenic separation or a membrane separation.

After the separation of the CO₂, the remaining gases 216, including ethane, ethylene, carbon monoxide, methane, and other gases, are fed to a hydrocarbon separation system 218. In the hydrocarbon separation system 218, cryogenic separation is used to separate the remaining gases 216 by boiling point. For example, the remaining gases 216 can be separated into a waste gas stream 220 that includes carbon monoxide, the ethylene 222, and a recycle ethane 224.

The recycle ethane 224 is then fed to the hydrate formation process 210. In the hydrate formation process 210, an ethane/water clathrate hydrate is formed as a solid, and is filtered from the acetic acid solution. This results in a more concentrated acetic acid solution 226. In various examples, the more concentrated acetic acid solution 226 is greater than about 50% acetic acid, greater than about 30% acetic acid, greater than about 15% acetic acid, or greater than about 10% acetic acid. The concentration of the more concentrated acetic acid solution 226 depends on the desired concentration for marketing the acetic acid, and on the inhibition of the clathrate hydrate formation in the more concentrated acetic acid solution 226.

The ethane/water clathrate hydrate that is separated from the more concentrated acetic acid solution 226 is melted in the hydrate formation process 210, forming the recycle ethane 208 that can be combined with the fresh ethane. This combined stream can then be fed to the ODH system 100 with the other feedstocks, such as the diluent 202 and the oxygen 204.

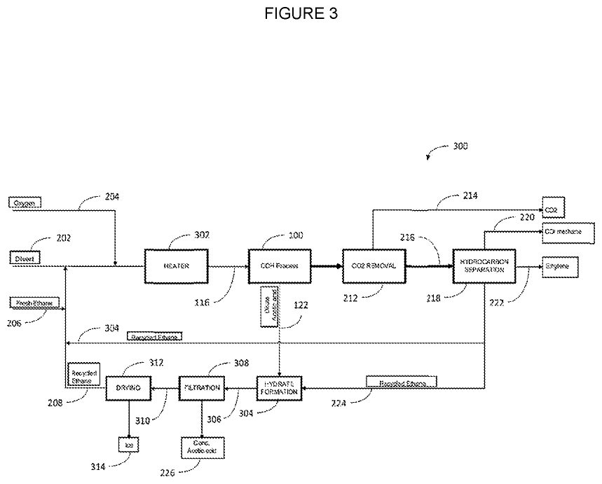

FIG. 3 is a schematic diagram of a process 300 for continuously forming hydrates to concentrate the acetic acid. Like numbered items are as described with respect to FIGS. 1 and 2. In this process 300, the feed streams 202, 204, and the combined fresh ethane 206 and recycle ethane 208, are passed through a heater 302 before being fed to the ODH system 100. The heater 302 may be placed immediately after a blending vessel that blends the components in water, and immediately before the ODH reactor, to limit the amount of time that the reactants are at reaction temperature before entering the ODH reactor.

In this process 300, the dilute acetic acid 122 is added to a hydrate formation vessel 304 along with the recycle ethane 224. The hydrate formation vessel 304 may be a continuous stirred tank reactor (CSTR), a vertical vessel, or another type of continuous reactor system. The solid hydrates formed in the hydrate formation vessel 304 are carried in a slurry 306 to a filtration system 308 to separate the solid hydrate 310 from the more concentrated acetic acid solution 226.

From the filtration system 308, the solid hydrates 310 are fed to a drying system 312. In the drying system 312, the solid hydrates 310 are melted to release the recycle ethane 208, which is then combined with the fresh ethane 206 to be used as a feed stream for the ODH system 100. As the temperature at which the separation occurs can be well below the freezing point of water, the water may be removed from the drying system 312 as ice 314.

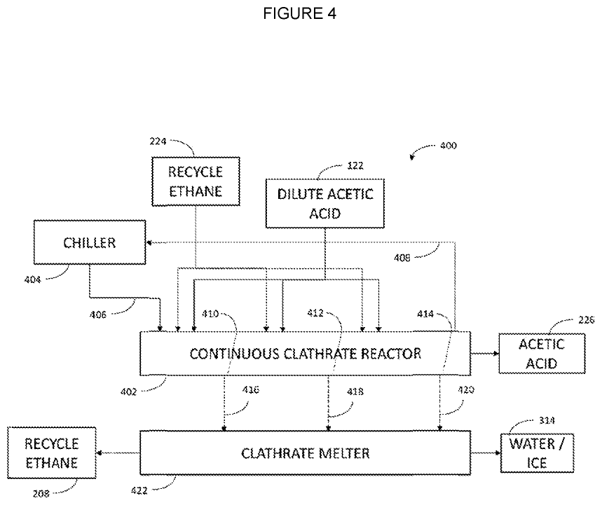

FIG. 4 is a schematic diagram of a process 400 for the concentration of acetic acid by the continuous formation and removal of clathrate hydrates. Like numbered items are as described with respect to the previous figures. In this figure, liquid flow is shown in solid lines, gas flow is shown in dotted lines, and solid flow is shown in dashed lines. In this process 400, only the materials and units involved in hydrate formation are shown.

In the process 400, the dilute acetic acid 122 and the recycle ethane 224 are added to a continuous clathrate reactor (CCR) 402 at a number of different injection points along the CCR 402. A chiller 404 is used to chill the CCR 402 to the temperature needed to form the solid hydrate, for example, below about 30° C., below about 20° C., below about 15° C., or below about 5° C. The chiller 404 can inject a chilled fluid 406 into heat exchangers in the CCR 402. The chilled fluid 406 can be ammonia, propane, hydrochlorofluorocarbons (HCFCs), or other fluids selected based on the operating temperature range. In the heat exchangers, the chilled fluid 406 can be flashed to a gas 408, which is returned to the chiller 404.

The operational temperature for the CCR 402 is selected based on the pressure, which is controlled by injection pumps (not shown). For example, the pressure can be 5000 kPa, 10,000 kPa, 15,000 kPa, 50,000 kPa, or higher. Higher pressures can be associated with the formation of hydrates at lower temperatures, but may be more difficult to control.

As the dilute acetic acid 122 and the recycle ethane 224 flow through the CCR 402, solid hydrates are formed from the water and ethane. In some embodiments, the solid hydrates are filtered from the flow in the CCR 402 by filters 410, 412, and 414.

The screen, or particle, size for solids to be removed by each of the filters can be selected based on the location in the CCR 402. As the acetic acid in the CCR 402 is more dilute at the beginning of the CCR 402, the formation of hydrates may be easier. Thus, the first filter 410 may be selected to remove larger particles of solid hydrate 416, allowing small hydrate particles, termed hydrate seeds, to pass through. The second filter 412 removes a further portion of the solid hydrate 418, allowing hydrate seeds to pass through into the final section of the CCR 402. The last filter 414 may be sized to remove most or all of the remaining solid hydrate 420. As described herein, depending on the desired concentration of the more concentrated acetic acid solution 226, the more concentrated acetic acid solution 226 may be reinjected into the CCR 402. In some examples, this would be performed after the first filter 410, so that the formation of hydrate seeds is not interrupted.

The solid hydrates 416, 418, and 420 are fed to a clathrate melter 422. In the clathrate melter, the temperature is raised, or the pressure is lowered, or both to allow the solid hydrates to degrade, releasing the recycle ethane 208 and the water or ice 314.

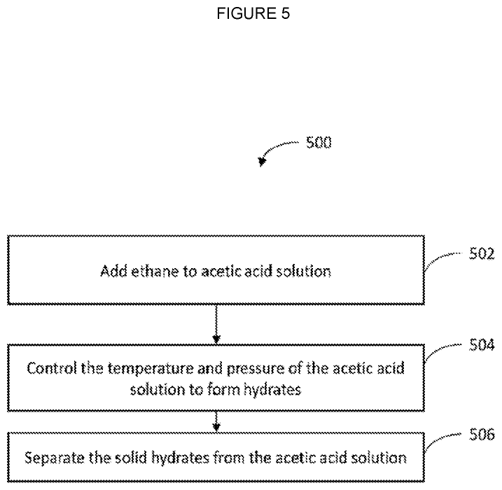

FIG. 5 is a process flow diagram of a method 500 for concentrating acetic acid using the formation of clathrate hydrates. The method 500 begins at block 502, with the addition of ethane to an acetic acid solution. As described above, the ethane and the acetic acid solution may be provided from an ODH reaction.

At block 504, the temperature and pressure of the acetic acid solution with the ethane is controlled to form hydrate solids. For example, as described herein, the pressure of the solution is raised and the temperature is lowered to a range that favors the formation of the hydrates.

At block 506, the solid hydrates are separated from the acetic acid solution. This increases the concentration of the acetic acid solution. As described herein, the hydrates can then be degraded to release the water and the ethane.

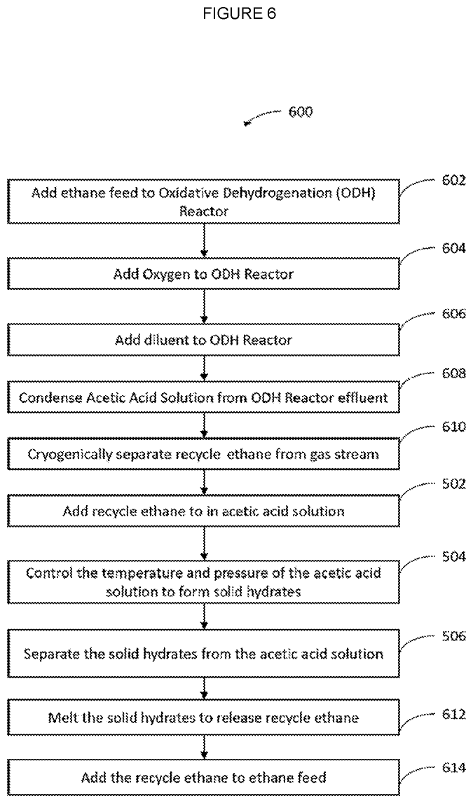

FIG. 6 is a process flow diagram of a method 600 for integrating the concentration of acetic acid using the formation of clathrate hydrates with an oxidative dehydrogenation process. Like numbered items are as described with respect to FIG. 5.

The method begins at block 602, with the addition of the ethane feed to the oxidative dehydrogenation (ODH) reactor. As described herein, the ethane feed may include recycle ethane from the melting of clathrate hydrates. At block 604, oxygen is added to the ODH reactor. At block 606, diluent is added to the ODH reactor. In some examples, the feed streams are blended and added to the ODH reactor at the same time, for example, using a water-filled blending vessel.

At block 608, a dilute acetic acid solution is condensed from the ODH reactor effluent, for example, in a quench tower, forming a gas stream. The heat from the condensation may be used in other portions of the process, for example, to melt or degrade clathrates or to heat feed streams for addition to the ODH reactor. In some examples, carbon dioxide is removed from the gas stream using an amine scrubber.

At block 610, recycle ethane is cryogenically separated from the gas stream. This may be done in a cryogenic separation tower, or by passing the gas stream through a cryogenic condenser.

As described with respect to FIG. 5, at block 502, the recycle ethane is added to the dilute acetic acid. At block 504, the temperature and pressure are controlled to form solid hydrates. At block 506, the solid hydrates are filtered from the acetic acid solution, resulting in a more concentrated acetic acid solution.

At block 612, the solid hydrates are melted to release the recycle ethane and water. The resulting water does not have to be fully melted, but may be in the form of ice. For example, as the pressure is dropped the solid hydrate may degrade forming ethane gas and ice.

At block 614, the recycle ethane is added to the ethane feed to the ODH reactor. For example, the recycle ethane may be blended with fresh ethane as needed.

### EXAMPLES

### Example 1a (ASPEN HYSYS Simulation for Feed Mixing)

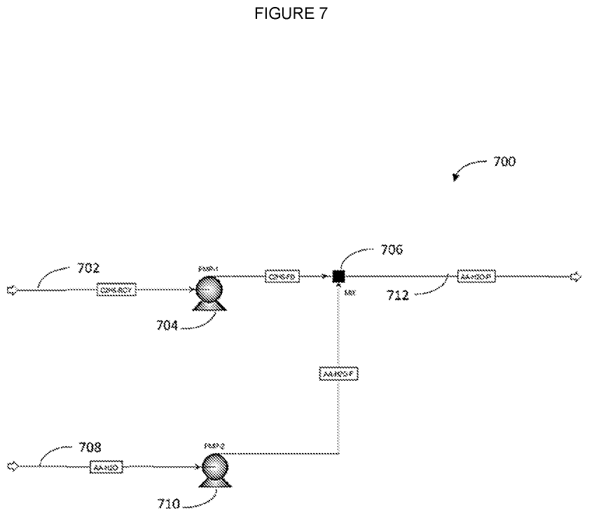

FIG. 7 is a simulation flowsheet 700 for simulating the mixing of the recycled ethane with the dilute acetic acid formation of clathrate hydrates of ethane using ASPEN HYSYS V11. In this example, the ethane 702 is pressurized by a first pump 704 then fed to a mixing vessel 706. Water 708 is fed to the mixing vessel 706 using a second pump 710. The hydrate content of resulting mixture 712 is then be calculated. For the simulation, the NRTL-HOC and STMNBS2 equations of state were used.

The ethane 702, for example, from the bottom of the hydrocarbon separator C2-splitter (C2H6-RCY) is at temperature and pressure of −9.8° C. and 1895 kPa respectively. The aqueous acetic acid stream (AA-H2O) is at temperature of 40° C. and a pressure of 142.3 kPa. These two streams were pumped separately to 17,000 kPa before mixing. The resulting mixed stream (AA-H2O-P) is at temperature and pressure of 35° C. and 17,000 kPa. The composition and conditions of this stream were then used for the hydrate formation calculation in CSMGem. Table 1 shows the mass and energy balances obtained from ASPEN Plus.

TABLE 1
Mass and Energy Balances

| | Units | AA-H2O | AA-H2O-P | AA-H2O-F | C2H6-FD | C2H6-RCY |
|---|---|---|---|---|---|---|
| From | | | MIX | PMP-2 | PMP-1 | |
| To | | PMP-2 | | MIX | MIX | PMP-1 |
| Temperature | C. | 40.0 | 35.0 | 42.3 | 6.9 | −9.8 |
| Pressure | kPa | 142.3 | 17000 | 17000 | 17000 | 1895 |
| Molar Liquid | | 1 | 1 | 1 | 1 | 1 |
| Fraction | | | | | | |
| Mass Density | kg/cum | 968.5 | 738.4 | 966.1 | 383.5 | 422.6 |
| Enthalpy Flow | GJ/hr | −3497.5 | −3869.9 | −3491.9 | −378.0 | −383.4 |
| Average MW | | 21.6 | 23.8 | 21.6 | 30.1 | 30.1 |
| Mole Flows | kmol/hr | 11599.1 | 15576.3 | 11599.1 | 3977.2 | 3977.2 |
| CH4 | kmol/hr | 0.0 | 0.0 | 0.0 | 0.0 | 0.0 |
| C2H4 | kmol/hr | 0.3 | 20.2 | 0.3 | 19.9 | 19.9 |
| C2H6 | kmol/hr | 0.2 | 3944.0 | 0.2 | 3943.8 | 3943.8 |
| C3H8 | kmol/hr | 0.0 | 13.5 | 0.0 | 13.5 | 13.5 |
| CH3COOH | kmol/hr | 987.5 | 987.5 | 987.5 | 0.0 | 0.0 |
| CO2 | kmol/hr | 0.0 | 0.0 | 0.0 | 0.0 | 0.0 |
| H2O | kmol/hr | 10611.1 | 10611.1 | 10611.1 | 0.0 | 0.0 |
| Mass Flows | kg/hr | 250479.4 | 370223.5 | 250479.4 | 119744.1 | 119744.1 |
| CH4 | kg/hr | 0.1 | 0.1 | 0.1 | 0.0 | 0.0 |
| C2H4 | kg/hr | 7.9 | 565.8 | 7.9 | 557.9 | 557.9 |
| C2H6 | kg/hr | 5.1 | 118594.5 | 5.1 | 118589.4 | 118589.4 |
| C3H8 | kg/hr | 0.0 | 596.8 | 0.0 | 596.8 | 596.8 |
| CH3COOH | kg/hr | 59303.4 | 59303.4 | 59303.4 | 0.0 | 0.0 |
| CO2 | kg/hr | 1.3 | 1.3 | 1.3 | 0.0 | 0.0 |
| H2O | kg/hr | 191161.7 | 191161.7 | 191161.7 | 0.0 | 0.0 |

### Example 1b (CSMGem Calculation for Hydrate Formation)

Hydrate formation calculation was performed using CSMGem V1.10. The feed used comprises ethane, ethylene, CO₂, and water. In this example, the calculation did not include acetic acid because it is not available in the software. However, it was assumed that acetic acid would not interfere with the kinetics of hydrate formation based on previous research. The feed composition for the calculation is similar to what is reported in Table 1 for the AA-H2O-P stream.

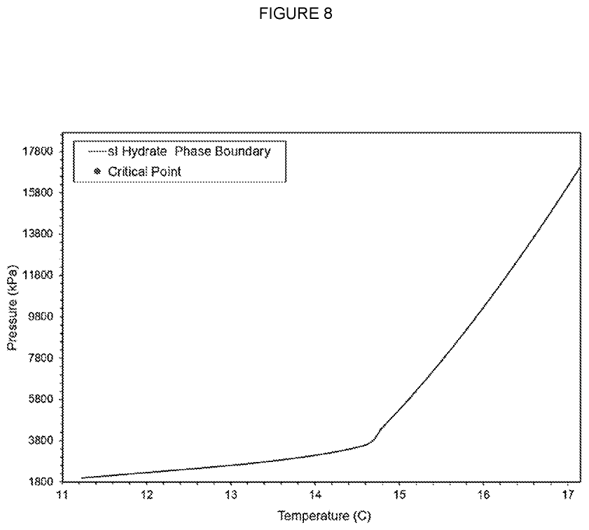

FIG. 8 is a plot of the resulting P-T diagram for the incipient hydrate formation. At 17° C. and 16,220 kPa, three phases were calculated to be present. This is shown in Table 2. The type 1 hydrate contains some of the ethane and ethylene; the CO₂ is almost negligible. The acetic acid, even though it was not included in the calculation, will be in the aqueous phase. Based on the calculation done outside the CSMGem, the concentration of the acetic acid was found to be 98.6 wt. %.

TABLE 2
Molar Composition of Phases Present for Hydrate Formation
Hydrate Formation P at T
Temperature = 17.0° C.
Pressure = 16223 kPa
Number of Phases Present: 3

| | Aqueous | Vapor | sI Hydrate |
|---|---|---|---|
| Ethylene | 0.000020 | 0.004420 | 0.000797 |
| Ethane | 0.001027 | 0.990851 | 0.114348 |
| Propane | 0.000001 | 0.004577 | 0.000001 |
| Water | 0.998952 | 0.000152 | 0.884853 |
| Phase Fraction | 0.185889 | 0.202009 | 0.612102 |

### Example 2a (ASPEN HYSYS Including Acetic Acid During Hydrate Formation)

Another hydrate calculation was performed using ASPEN HYSYS. The concentration of acetic acid used for these simulations was about 16 wt. %. This was to determine the effects of acetic acid on the formation of hydrates. In Examples 1a and 1b, the hydrate calculation was done using CSMGem, which does not include acetic acid. The simulation was repeated in ASPEN HYSYS using the same feed composition, and the CPA property package. Hydrate formation was found at 17.01° C. and 16,220 kPa. Further calculations indicated that hydrates are also formed at 17.08° C. and 16,220 kPa or 17° C. and 15820 kPa.

### Example 2b (ASPEN HYSYS Including Acetic Acid During Hydrate Formation)

When the concentration of acetic acid was decreased to 8 wt. % at 17.02° C. and 16,220 kPa, hydrates were formed. Further simulations showed that hydrates are also formed at 17.41° C. and 16220 kPa or 17° C. and 14,000 kPa.

### Example 2c (ASPEN HYSYS Including Acetic Acid During Hydrate Formation)

For acetic acid concentration of 32 wt. % in the feed to the hydrate formation unit at 16.99° C. and 16,220 kPa, no hydrate was predicted to be formed. However, hydrates are predicted to be formed at 16.70° C. and 16220 kPa.

Based on these simulations, the acetic acid concentration may be specified to be <32 wt. % for hydrate formation. This range is still within the acetic concentration from the ODH process. Further, as the acetic acid may be a kinetic hydrate inhibitor, the use of the continuous process described with respect to FIG. 4 may allow the formation of hydrate seeds at lower concentrations of acetic acid, which can continue the formation process at higher concentrations.

### Example 3 (ASPEN HYSYS Including Acetic Acid During Hydrate Formation)

Another simulation was run test the possibility of generating the hydrate using the bottom stream pressure from the hydrocarbon separator. Using this stream, avoids the need to pump the ethane stream from the hydrocarbon separator into the hydrate formation unit.

Using this pressure, and an acetic acid concentration of 16 wt. % in the feed and stream conditions of 17.42° C. and 1895 kPa, no hydrates are formed. However, hydrates are found to be formed at 11.32° C. and 1895 kPa or 17.42° C. and 18,300 kPa. Interestingly, at a temperature of 11.32° C. and a pressure at 1895 kPa, there is vapor in the equilibrium phase. This allows vaporization of the liquid ethane from the hydrocarbon separator without the need for a heat exchanger.

### IMPLEMENTATIONS

An implementation described in examples herein provides a method for concentrating acetic acid. The method includes adding ethane to an acetic acid solution, controlling temperature and pressure to form a mixture of solid hydrates in the acetic acid solution, and separating the solid hydrates from the acetic acid solution to form a more concentrated acetic acid solution.

In an aspect, the ethane includes recycle ethane from an ethane oxidative dehydrogenation process.

In an aspect, wherein the acetic acid solution includes acetic acid from an ethane oxidative dehydrogenation process.

In an aspect, the method includes melting the solid hydrates to separate at least a portion of the ethane from the solid hydrates.

In an aspect, the method includes adding at least a portion of the ethane separated from the solid hydrates to a feed stream for an oxidative dehydrogenation reactor. In an aspect, the method includes forming the mixture of solid hydrates in a continuous process.

In an aspect, the method includes filtering larger particles of solid hydrates from the acetic acid solution, and allowing smaller particles of solid hydrates to stay in the solution as hydrate seeds.

In an aspect, the method includes adding ethane to an oxidative dehydrogenation (ODH) reactor, adding oxygen to the ODH reactor, adding a diluent to the ODH reactor, and cooling an effluent stream including ethylene, ethane, steam, and acetic acid to form the acetic acid solution and a gas effluent stream.

In an aspect, the method includes separating carbon dioxide from the gas effluent stream to form a non-condensable effluent stream.

In an aspect, the method includes adding the acetic acid solution to a hydrates reactor.

In an aspect, the method includes cryogenically separating recycle ethane from the gas effluent stream.

In an aspect, the method includes adding the recycle ethane to the hydrates reactor.

In an aspect, the method includes cryogenically separating ethylene from the gas effluent stream.

Another implementation described in examples herein provides a system for concentrating an acetic acid solution. The system includes a hydrate reactor to form solid hydrates in the acetic acid solution, a filtration system to remove the solid hydrates formed in the hydrate reactor, and a drying system to separate ethane from water.

In an aspect, the system includes an oxidative dehydrogenation reactor to form acetic acid and steam, and a quench tower to condense the acetic acid and steam from an effluent of the oxidative dehydrogenation reactor form the acetic acid solution.

In an aspect, the system includes a carbon dioxide removal system to remove carbon dioxide after the acetic acid solution is condensed from the effluent.

In an aspect, the system includes a hydrocarbon separation system to separate ethane from the effluent.

In an aspect, the hydrate reactor includes a continuous reactor vessel.

In an aspect, the continuous reactor vessel includes at least two injection points for the acetic acid solution and at least two injection points for ethane.

Other implementations are also within the scope of the following claims.

1. A method for concentrating acetic acid, comprising:
adding ethane to an acetic acid solution;
controlling temperature and pressure to form a mixture of solid hydrates in the acetic acid solution; and
separating the solid hydrates from the acetic acid solution to form a more concentrated acetic acid solution.

2. The method of claim 1, wherein the ethane comprises recycle ethane from an ethane oxidative dehydrogenation process.

3. The method of claim 1, wherein the acetic acid solution comprises acetic acid from an ethane oxidative dehydrogenation process.

4. The method of claim 1, further comprising melting the solid hydrates to separate at least a portion of the ethane from the solid hydrates.

5. The method of claim 4, comprising adding at least a portion of the ethane separated from the solid hydrates to a feed stream for an oxidative dehydrogenation reactor.

6. The method of claim 1, comprising forming the mixture of solid hydrates in a continuous process.

7. The method of claim 6, comprising:
filtering larger particles of solid hydrates from the acetic acid solution; and
allowing smaller particles of solid hydrates to stay in the solution as hydrate seeds.

8. The method of claim 1, comprising:
adding ethane to an oxidative dehydrogenation (ODH) reactor;
adding oxygen to the ODH reactor;
adding a diluent to the ODH reactor; and
cooling an effluent stream comprising ethylene, ethane, steam, and acetic acid to form the acetic acid solution and a gas effluent stream.

9. The method of claim 8, comprising separating carbon dioxide from the gas effluent stream to form a non-condensable effluent stream.

10. The method of claim 8, comprising adding the acetic acid solution to a hydrates reactor.

11. The method of claim 10, comprising cryogenically separating recycle ethane from the gas effluent stream.

12. The method of claim 11, comprising adding the recycle ethane to the hydrates reactor.

13. The method of claim 8, comprising cryogenically separating ethylene from the gas effluent stream.

14. A system for concentrating an acetic acid solution, comprising:
a hydrate reactor having a first inlet configured to receive acetic acid and a second inlet configured to receive ethane;
a chiller;
a pump, wherein the chiller and the pump are configured to control the temperature and pressure so that, when water, acetic acid and ethane are present in the hydrate reactor, the water and ethane form solid hydrates in an acetic acid solution;
a filtration system to remove the solid hydrates formed in the hydrate reactor; and
a drying system to separate the ethane from the water.

15. The system of claim 14, comprising:
an oxidative dehydrogenation reactor to form acetic acid and steam; and
a quench tower to condense the acetic acid and steam from an effluent of the oxidative dehydrogenation reactor form the acetic acid solution.

16. The system of claim 15, comprising a carbon dioxide removal system to remove carbon dioxide after the acetic acid solution is condensed from the effluent.

17. The system of claim 15, comprising a hydrocarbon separation system to separate ethane from the effluent.

18. The system of claim 14, wherein the hydrate reactor comprises a continuous reactor vessel.

19. The system of claim 18, wherein the continuous reactor vessel comprises at least two injection points for the acetic acid solution and at least two injection points for ethane.
